(19) United States
(12) Patent Application Publication
(10) Pub. No.: US 2011/0046459 A1
(43) Pub. Date: Feb. 24, 2011

(54) Non-Invasive Patient Monitoring Using Near Infrared Spectrophotometry

(75) Inventors: Wei ZHANG, Union City, CA (US); Zengpin YU, Palo Alto, CA (US); Thomas P. NEFF, Newark, CA (US); Shih-Ping WANG, Los Altos, CA (US)

Correspondence Address: COOPER & DUNHAM, LLP, 30 Rockefeller Plaza, 20th Floor, NEW YORK, NY, 10112, US

(73) Assignee: O2 MEDTECH, INC., Los Altos, CA (US)

(21) Appl. No.: 12/826,218

(22) Filed: Jun. 29, 2010

Related U.S. Application Data
(63) Continuation-in-part of application No. 12/815,696, filed on Jun. 15, 2010.
(60) Provisional application No. 61/222,099, filed on Jun. 30, 2009.
(60) Provisional application No. 61/187,222, filed on Jun. 15, 2009.
(60) Provisional application No. 61/255,851, filed on Oct. 28, 2009.

(51) Int. Cl. A61B 5/00

(52) U.S. Cl. 600/310

(57) ABSTRACT

Methods, systems, and related computer program products for non-invasive spectrophotometric monitoring of an optical property of a medium are described. Respective light portions are propagated through respective at least partially non-overlapping subregions of the medium during each of a calibration interval and a subsequent monitoring interval. Detections of the light portions acquired during the calibration interval are processed to compute at least one algorithm compensation that causes a first result related to the optical property for the first subregion to be substantially equal to a second result related to the optical property for the second subregion. Subsequently, detections of respective light portions acquired during the monitoring interval are processed in conjunction with the at least one algorithm compensation to compute a monitoring result for the optical property. Advantages can include a reduction in the number of sources/detectors required and/or an obviation of symmetry requirements in source/detector layout.

### CROSS-REFERENCE TO RELATED APPLICATIONS

This patent application claims the benefit of U.S. Provisional Ser. No. 61/222,099, filed Jun. 30, 2009. This patent application is a continuation-in-part of U.S. Ser. No. 12/815,696, filed Jun. 15, 2010 (Atty. Dkt. 6949/81719), which claims the benefit of U.S. Provisional Ser. No. 61/187,222, filed Jun. 15, 2009. This patent application also claims the benefit of U.S. Provisional Ser. No. 61/255,851, filed Oct. 28, 2009. Each of the above-referenced patent applications is incorporated by reference herein.

### FIELD

This patent specification relates to the non-invasive monitoring of a physiological condition of a patient using information from near-infrared (NIR) optical scans. More particularly, this patent specification relates to systems, methods, and related computer program products for the non-invasive NIR spectrophotometric (NIRS) monitoring of one or more chromophore levels, such as oxygenated hemoglobin levels, in one or more parts of the patient anatomy, such as the human brain.

### BACKGROUND AND SUMMARY

The use of near-infrared (NIR) light as a basis for the measurement of biological properties or conditions in living tissue is particularly appealing because of its relative safety as compared, for example, to the use of ionizing radiation. Various techniques have been proposed for non-invasive NIR spectroscopy or NIR spectrophotometry (NIRS) of biological tissue. Generally speaking, these techniques are directed to detecting the concentrations of one or more chromophores in the biological tissue, such as blood hemoglobin in oxygenated (HbO) and deoxygenated (Hb) states.

As used herein, NIRS tissue oxygenation level monitoring refers to the introduction of NIR radiation (e.g., in the 500-2000 nm range) into a tissue volume and the processing of received NIR radiation migrating outward from the tissue volume to generate at least one metric indicative of oxygenation level(s) in the tissue. One example of an oxygenation level metric is oxygen saturation [SO2], which refers to the fraction or percentage of total hemoglobin [HbT] that is oxygenated hemoglobin [HbO]. NIRS-based oxygen saturation readings can be classified as “relative” in nature (i.e., presented only in terms of their change over time) or can be “absolute” in nature (i.e., computed from absolute concentrations of [HbO] and [HbT] in units of grams per deciliter (g/dl) or equivalent). As will be appreciated by a person skilled in the art in view of the present disclosure, the preferred embodiments described further hereinbelow are applicable for systems that acquire absolute readings, relative readings, or both.

NIRS cerebral oxygenation level monitoring, which refers to the transcranial introduction of NIR radiation into the intracranial compartment and the processing of received NIR radiation migrating outward therefrom to generate at least one metric indicative of oxygenation level(s) in the brain, represents one particularly important type NIR tissue oxygenation level monitoring. One exemplary need for reliable determination of oxygen saturation levels in the human brain arises in the context of the millions of surgical procedures performed under general anesthesia every year. One statistic recited in U.S. 5902235 is that at least 2,000 patients die each year in the United States alone due to anesthetic accidents, while numerous other such incidents result in at least some amount of brain damage. Certain surgical procedures, particularly of a neurological, cardiac or vascular nature, may require induced low blood flow or pressure conditions, which inevitably involves the potential of insufficient oxygen delivery to the brain. Many surgical procedures also involve the possibility that a blood clot or other clottable material can break free, or otherwise get introduced into the bloodstream, and travel to the brain to cause a localized or widespread ischemic event therein. At the same time, the brain is highly intolerant to oxygen deprivation, and brain cells will die (become infarcted) within a few minutes if not sufficiently oxygenated. Accordingly, the availability of immediate, accurate and reliable information concerning brain oxygenation levels is of critical importance to anesthesiologists and surgeons, as well as other involved medical practitioners.

Pulse oximetry, in which infrared sources and detectors are placed across a thin part of the patient's anatomy such as a fingertip or earlobe, has arisen as a standard of care for all operating room procedures. However, pulse oximetry provides only a general measure of blood oxygenation as represented by the blood passing by the fingertip or earlobe sensor, and does not provide a measure of oxygen levels in vital organs such as the brain. In this sense, the surgeons in the operating room essentially “fly blind” with respect to brain oxygenation levels, which can be a major source of risk for patients (e.g., stroke) as well as a major source of cost and liability issues for hospitals and medical insurers.

Valid NIRS cerebral oxygenation level readings provide crucial monitoring data for the surgeon and other attending medical personnel, providing more direct data on brain oxygenation levels than pulse oximeters while being just as safe and non-invasive as pulse oximeters. Generally speaking, such systems involve the attachment of one or more NIR probe patches to the forehead or other available skin surface of the head. Each NIR probe patch usually comprises one or more NIR optical source ports for introducing NIR radiation into the cerebral tissue and one or more NIR optical receiver ports for detecting NIR radiation that has migrated through at least a portion of the cerebral tissue. One or more oxygenation level metrics are then provided on a viewable display in a digital readout and/or graphical format.

One desirable attribute of a NIRS cerebral oxygenation level detection system, also termed a NIRS cerebral oximeter, is the ability to provide bilateral output readings, e.g., output readings that are separately applicable to the left and right hemispheres of the brain or, more generally, to a left lateral region versus a right lateral region of the head relative to some dividing point, line, or plane. Such bilaterality can provide crucial information to the clinician regarding which side of the brain (if any) is experiencing an ischemic condition.

For NIRS oximeters more generally, it is desirable to be able to provide, for some region of biological tissue in the head or another part of the anatomy, two or more output readings that are at least partially localized with respect to each other, i.e., two or more output readings that are relevant to two or more respective regions of biological tissue that are at least partially non-overlapping with each other. Such localized readings can yield particular insights, such as the manner of onset of an ischemic condition, when acquired and compared over time in a patient monitoring session. By way of further example, where the different readings relate to a common tissue region but are localized to encompass different tissue depths (e.g., by virtue of different source-detector spacings such that the subsurface “banana” shaped regions encompass different tissue depths), the localized readings can be processed as described in the commonly assigned U.S. Ser. No. 12/815,696, supra, to yield layer-specific output readings.

It would be desirable to provide a cerebral oximetry system that provides oxygenation level monitoring (relative or absolute) that is bilateral, while also providing for greater penetration distance into the brain. It would be further desirable to provide such bilateral oxygenation level monitoring in a cerebral oximetry system having fewer required source/detector hardware elements, while still obviating source intensity/detector efficiency differences and/or coupling efficiency differences among the different sources and detectors. It would be further desirable to provide such a bilateral cerebral oximetry system having NIR probe patches that are more stable upon the forehead, more comfortable for the patient, less expensive to fabricate, and that are easier to connect and disconnect from the patient.

More generally, it would be desirable to provide methods and systems for non-invasive NIRS tissue monitoring, for the head or other part of the anatomy, that can provide localized output readings using fewer required source/detector hardware elements, optionally at relatively deep penetration levels relative to the tissue surface on which the sources and detectors are placed, while obviating source intensity/detector efficiency differences and/or coupling efficiency differences among the different sources and detectors based on clinically realistic assumptions about the patient monitoring session. One or more other issues arises in the implementation of NIRS oximetry systems that is at least partially addressed by one or more of the preferred embodiments described further hereinbelow. By way of example, U.S. Pat. No. 6,078,833, which is incorporated by reference herein, discusses cancellation of source intensity/coupling efficiency and detector efficiency/coupling efficiency factors by ensuring particular symmetries in the spatial layout of the sources and detectors across the tissue surface. For various practical, clinical, manufacturing-related, and/or business-related issues it may be desirable to avoid the need for such source-detector symmetries to be present, while still obviating the effects of source intensity/detector efficiency differences and/or coupling efficiency differences among the different sources and detectors.

By way of further example, another issue that arises in NIR cerebral oximetry (as well as more generally for NIR oximetry for other part of the patient anatomy) relates to the particular selection of information that is displayed to the clinician and the way that information is displayed to the clinician. As with many medical instrumentation modalities, there is a tension that arises between (i) the amount of information that is available for display to the clinician, and (ii) the ability of the clinician to quickly and effectively perceive the displayed information. As with many medical instrumentation modalities, a balance must be found between displaying too little information and too much information, keeping in mind that even the “right amount” of information can become “too much information” if it is not judiciously arranged on the viewer display. However, NIR cerebral oximetry brings about additional issues that even further complicate and exacerbate these information display issues. The additional issues include (iii) the varying degrees of reliability and accuracy that may be associated with the NIR cerebral oximeter outputs at any particular interval during the patient monitoring session, (iv) how an NIR cerebral oximeter might intrinsically detect intervals in which the output readings might not be reliable or accurate, (v) the question of whether, when, and how often the NIR cerebral oximeter should remind the clinician that the displayed readings might not be reliable or accurate at a particular detected interval, and (vi) displaying the additional reliability/accuracy reminders to the clinician in a manner that allows for quick and effective perception of both the NIR cerebral oximeter outputs and the additional reliability/accuracy reminders. Even ostensibly subtle changes to the information selection and presentation strategies of an NIR cerebral oximeter user interface can be determining factors in the overall desirability, effectiveness, and marketability of that NIR cerebral oximeter. Other issues arise as would be apparent to one skilled in the art upon reading the present disclosure.

It is to be appreciated that although one or more preferred embodiments is detailed hereinbelow in the particular context of NIR cerebral oxygenation level monitoring (NIR cerebral oximetry), the present teachings are readily applicable to the non-invasive spectrophotometric monitoring of any of a variety of different body parts in which relatively deep tissue readings are desired on a spatially differentiated basis over time including, but not limited to, the kidney, lung, and liver, and furthermore are applicable for the monitoring of any of a variety of different chromophore types therein.

Provided according to one or more preferred embodiments are methods, systems, and related computer program products for non-invasive spectrophotometric monitoring of an optical property of a medium. A first optical source, a second optical source, a first optical detector, and a second optical detector are secured to a surface of the medium. During each of a calibration interval and a monitoring interval, the monitoring interval being subsequent to the calibration interval, a first portion of light is propagated from the first optical source through the medium to the first optical detector, a second portion of light is propagated from the second optical source through the medium to the first optical detector, a third portion of light is propagated from the first optical source through the medium to the second optical detector, and a fourth portion of light is propagated from the second optical source through the medium to the second optical detector. Detections of the first, second, third, and fourth light portions acquired during the calibration time interval are processed to compute at least one algorithm compensation that causes a first result related to the optical property based on the first and second detected light portions to be substantially equal to a second result related to the optical property based on the third and fourth detected light portions. Subsequent to the calibration interval, detections of the first, second, third, and fourth light portions acquired during the monitoring time interval are processed in conjunction with the at least one algorithm compensation to compute a monitoring result for the optical property of the medium. In another preferred embodiment the sources and detectors are interchanged, wherein the second portion of light is propagated from the first light source through the medium to the second detector and the third portion of light is propagated from the second light source through the medium to the first detector.

One or more advantages is provided in spectrophotometric monitoring according to one or more of the preferred embodiments including, but not limited to, a capability for providing spatially localized output readings using fewer required source/detector hardware elements than is required in one or more prior art scenarios, while at least partially obviating source intensity/detector efficiency differences and/or coupling efficiency differences among the different sources and detectors based on clinically realistic assumptions about the patient monitoring session. The one or more advantages further includes an ability to provide such obviation of source intensity/detector efficiency differences and/or coupling efficiency differences among the different sources and detectors without requiring particular spatial symmetries in the layout of the sources and detections as is required in one or more prior art scenarios.

### BRIEF DESCRIPTION OF THE DRAWINGS

FIG. 1 illustrates a prior art bilateral cerebral spectrophotometric monitoring system;

FIGS. 2A-2C illustrate a prior art slope method used in spectrophotometric monitoring;

FIGS. 3A-3C illustrate equations used in particular prior art phase modulated spectrophotometric (PMS) and continuous wave (CW) spectrophotometric monitoring scenarios;

FIG. 4 illustrates a prior art arrangement of non-ideal optical sources and detectors on a probe patch;

FIG. 5 illustrates an intensity-based slope computation based on a prior art symmetric source-detector layout;

FIG. 6 illustrates an intensity-based slope computation based on a prior art symmetric source-detector layout;

FIG. 7A illustrates a near-infrared spectrophotometric (NIR) cerebral oximeter according to a preferred embodiment;

FIG. 7B-1 illustrates an NIR probe patch according to a preferred embodiment;

FIG. 7B-2 illustrates exemplary dimensions associated with the NIR probe patch of FIG. 7B-1;

FIG. 7C illustrates an NIR probe patch according to a preferred embodiment;

FIGS. 7D-1 and 7D-2 illustrate an NIR probe patch according to a preferred embodiment;

FIG. 7E illustrates the NIR probe patch of FIG. 7D-1 as applied to a surface of a biological volume according to a preferred embodiment;

FIG. 7F illustrates dual instances of the NIR probe patch of FIG. 7D-1 as applied to a forehead of a patient for bilateral cerebral oximetry according to a preferred embodiment;

FIG. 8A illustrates near-infrared spectrophotometric (NIRS) monitoring of a biological volume of a patient according to a preferred embodiment;

FIG. 8B illustrates an alternative version of the probe patch illustrated in FIG. 8A according to a preferred embodiment;

FIGS. 9A-9C illustrate equations for adapting a slope method for NIRS monitoring of a biological volume according to a preferred embodiment;

FIG. 10 illustrates NIRS monitoring of a biological volume of a patient according to a preferred embodiment;

FIG. 11A illustrates an NIR probe patch according to a preferred embodiment;

FIG. 11B illustrates exemplary dimensions associated with the NIR probe patch of FIG. 11A;

FIGS. 12, 13, and 14A each illustrate an NIR probe patch according to a preferred embodiment;

FIG. 14B illustrates exemplary dimensions associated with the NIR probe patch of FIG. 14A;

FIG. 15 illustrates an NIR probe patch according to a preferred embodiment;

FIG. 16 illustrates an NIR cerebral oximetry system according to a preferred embodiment;

FIGS. 17-18 each illustrate an NIR probe patch according to a preferred embodiments;

FIG. 19 illustrates measurement and computation of near, far, and deep spectrophotometric readings for a two-layer tissue volume according to a preferred embodiment;

FIG. 20 illustrates a deep-layer spectrophotometric computation model according to a preferred embodiment; and

FIGS. 21, 22, 23A-B, 24, 25, 26, 27A-B, 28, and 29 illustrate examples of user displays for NIRS spectrophotometric monitoring systems according to one or more preferred embodiments.

### DETAILED DESCRIPTION

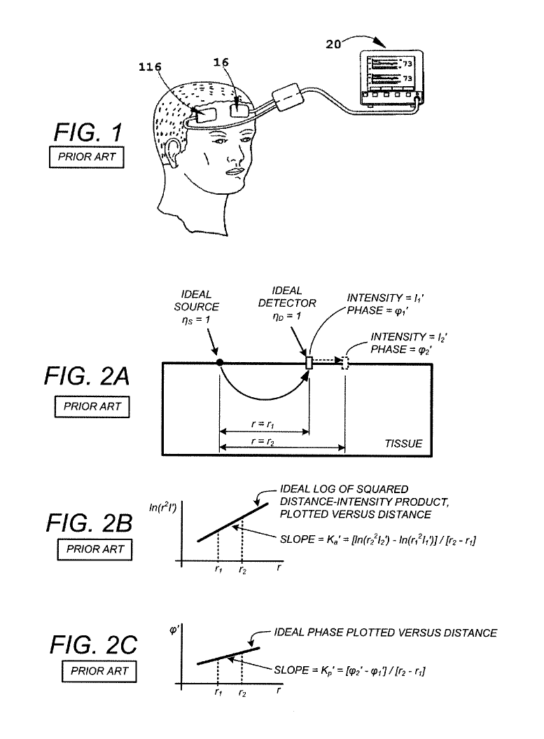

FIG. 1 illustrates a prior art proposal for a bilateral monitoring system in which two NIR probe patches 16 and 116 are placed on the forehead of the patient. The prior art proposal of FIG. 1 is further described in U.S. Pat. No. 6,615,065, which is incorporated by reference herein. Separate readings for the left and right sides of the brain are acquired and displayed separately on an output display 20. As illustrated in the proposal of FIG. 1, NIR probe patches are often placed on the forehead of the patient. The forehead represents a generally desirable region for attaching NIR probe patches, for at least the reason that the forehead is generally free of hair follicles. Even for a smoothly shaved head, the presence of hair follicles can introduce substantial amounts of noise and other interference into the NIR signals.

However, the use in FIG. 1 of two separate NIR probe patches on the forehead is antagonistic to an even more important goal of NIR cerebral oximetry, which is to obtain “deep” readings that are relevant to the brain tissue, rather than to the intervening skin, scalp, skull, dura, and cerebrospinal fluid (CSF) tissue. According to one thumbnail estimate provided in U.S. Pat. No. 5,853,370, which is incorporated by reference herein, the average penetration depth for a NIRS source-detector pair is about one-half of the lateral separation between the source and the detector. Because the source and the detector for any particular source-detector pair are required to be present on the same NIR probe patch (due to the need for precise, predetermined source-detector separation distances), the maximum source-detector separation distance for the prior art proposal of FIG. 1 is limited by the spatial extent of each individual NIR probe patch 16 and 116. Moreover, the use in FIG. 1 of two separate NIR probe patches on the forehead also brings about the need for left-right duplication of multiple source-detector pairs in order to obviate source intensity differences, detector efficiency differences, and skin coupling efficiency differences among the sources and detectors116.

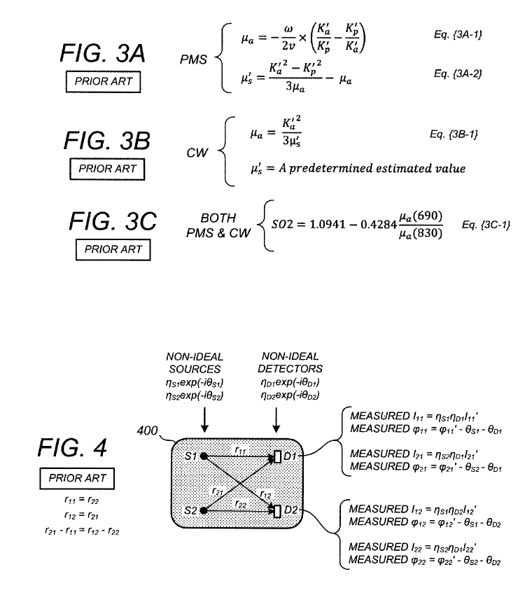

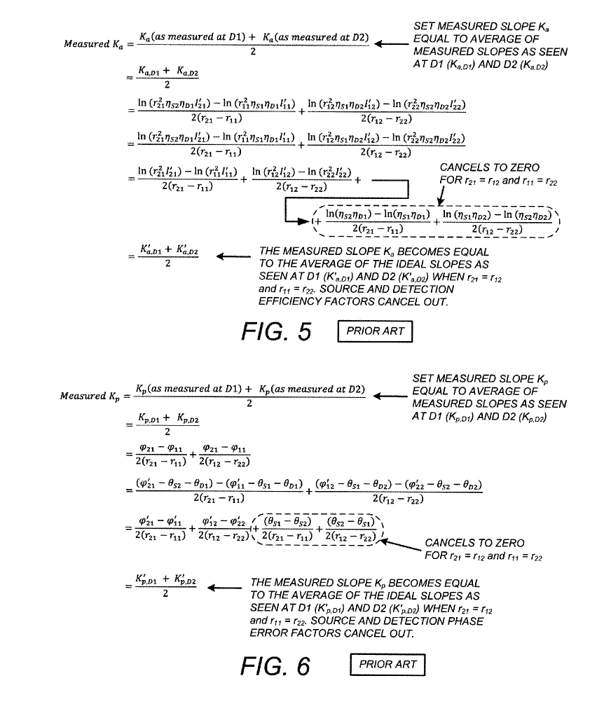

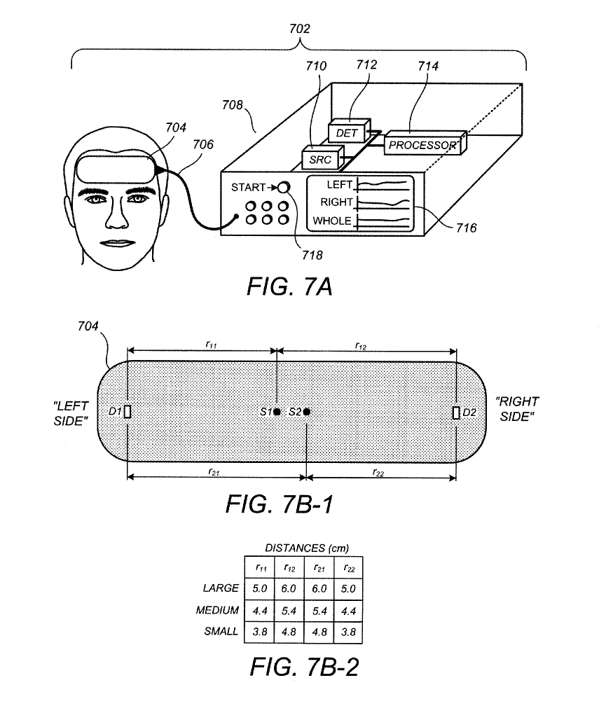

FIGS. 2A-2C illustrate a prior art slope method used in spectrophotometric monitoring. FIGS. 3A-3C illustrate equations used in particular prior art phase modulated spectrophotometric (PMS) and continuous wave (CW) spectrophotometric monitoring scenarios. Summarized in FIGS. 2A-2C and 3A-3C is the well-accepted “slope method” for computing tissue oxygenation levels (see, e.g., Fantini, Franceschini, and Gratton, “Semi-Infinite-Geometry Boundary Problem For Light Migration In Highly Scattering Media: A Frequency-Domain Study In The Diffusion Approximation,” J. Opt. Soc. Am. B, Vol. 11, pp. 2128-38 (1994) and Fantini, Hueber, and Franceschini, et. al., “Non-Invasive Optical Monitoring of the Newborn Piglet Brain Using Continuous-Wave and Frequency-Domain Spectroscopy,” Phys. Med. Biol., Vol. 44, pp. 1543-1563 (1999), each of which is incorporated by reference herein), while FIGS. 4-6 set forth one known method (see, e.g., U.S. Pat. No. 6,078,833, which is incorporated by reference herein) for using multiple source-detector pairs positioned over a common region to obviate source intensity differences, detector efficiency differences, and skin coupling efficiency differences among the sources and detectors.

Notationally, the prime symbol (′) is used to denote ideal intensities (I′) and ideal phases (φ′) that would result from ideal sources and ideal detectors (including ideal skin coupling), as well as ideal slopes (K′) of any plotted functions based on those ideal intensities and phases. In contrast, non-primed versions of those quantities refer to the physically measured versions of those values in the real world, and are termed herein measured intensities (I) and measured phases (φ), as well as measured slopes (K) of the plotted functions based on the measured intensities and measured phases. For PMS (phase modulated spectrophotometry) systems, also termed frequency domain spectrophotometry systems, the basis of the slope method is that for any particular NIR radiation wavelength, a plot of log (r²I′) versus r (where r is the source-detector distance) (FIG. 2B) has a relatively constant slope Kₐ′ over an appreciably useful range of distances, a plot of φ′ versus r (FIG. 2C) also has a relatively constant slope Kₚ′ over an appreciably useful range of distances, and the values of Kₐ′ and Kₚ′ can be used to compute the absorption coefficient μₐ (FIG. 3A, Eq. {3A-1}) and the effective or reduced scattering coefficient μₛ′ (FIG. 3A, Eq. {3A-2}) for that NIR radiation wavelength, where ω is the angular frequency corresponding to the source intensity modulation and v is the speed of light in the tissue. For CW (continuous wave) spectrophotometry systems in which there is no high-frequency modulation or phase measurements, the value of Kₐ′ can be used to compute the absorption coefficient μₐ (FIG. 3B, Eq. {3B-1}) for that NIR radiation wavelength using a fixed estimate of the effective scattering coefficient μₛ′. Based on the absorption coefficient μₐ for multiple NIR wavelengths (on opposite sides of the isosbestic wavelength for oxygenated and deoxygenated hemoglobin) the oxygenated hemoglobin saturation value SO2 is then readily determined, with {Eq. 3C-1} setting forth the formula for the particular NIR wavelengths of 690 nm and 830 nm. Generally speaking, the SO2 reading for the PMS-based measurements can be characterized as an absolute percentage value, whereas the SO2 reading for CW measurements should be taken only as a relative value over time.

FIG. 4 illustrates a prior art arrangement of non-ideal optical sources and detectors on a probe patch. As illustrated in FIG. 4, a non-ideal source S can be modeled as an ideal source as modified by a complex coefficient η_{S}exp(−iθ_{S}), which is termed herein the source intensity/coupling coefficient. For simplicity of nomenclature, although the magnitude η_{S} is more generally associated with variations in both source intensity and skin coupling, the magnitude η_{S} is simply referenced herein as “source coupling efficiency.” The phase term θ_{S} is referred to herein as the “source phase error.” Likewise, as illustrated in FIG. 4, a non-ideal detector D can be modeled as an ideal detector as modified by a complex coefficient η_{D}exp(−iθ_{D}), which is termed herein the detector sensitivity/coupling coefficient. For simplicity of nomenclature, although the magnitude η_{D} is more generally associated with variations in both detector sensitivity and skin coupling, the magnitude η_{D}, is simply referenced herein as “detector coupling efficiency.” The phase term θ_{D} is referred to herein as the “detector phase error.”

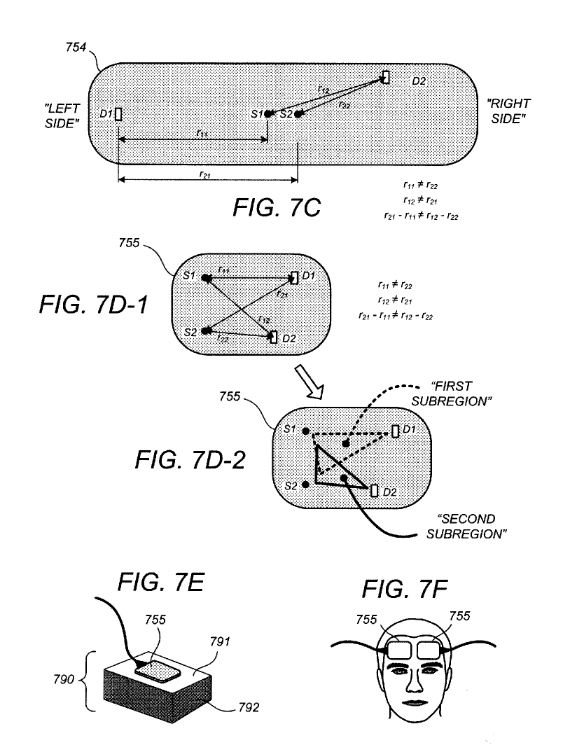

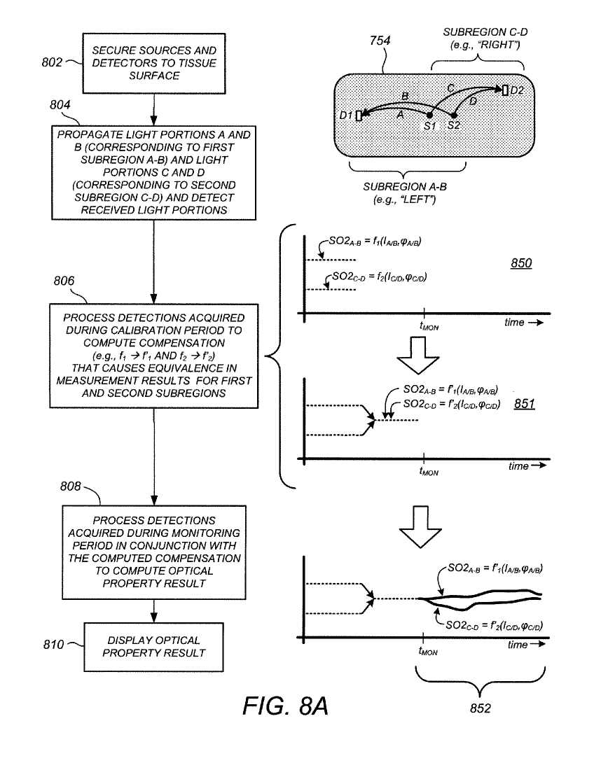

FIG. 5 illustrates an intensity-based slope computation based on a symmetric source-detector layout according to the prior art. FIG. 6 illustrates an intensity-based slope computation based on a symmetric source-detector layout according to the prior art. For simplicity and clarity of explanation, the more general case of PMS modulation is detailed further herein, with it being understood that CW methods would be analogous except with omitted phase factors and omitted phase-related slope computations. In the event that a real-world source S1 (and real-world source-skin coupling) was used and two real-world detectors D1 and D2 (with real-world detector-skin coupling) were positioned at r1 and r2, respectively, in the configuration of FIG. 2A, it could readily be shown that the values of μₐ and μₛ′ would include unknown coupling efficiency and phase error factors in addition to the known measured intensities I₁₂ and I₂₂. Because the coupling efficiency and phase error factors are unknown, the values of μₐ and μₛ′ would either be non-determinable, or else broad assumptions regarding coupling efficiency and phase error factors would need to be made. However, as summarized in FIGS. 4-6 and described further in U.S. Pat. No. 6,078,833, supra, the presence of different coupling efficiencies can be obviated by (i) adding a second source S2, (ii) positioning the two sources S1, S2 and two detectors D1, D2 in a symmetric relationship such that r₂₁=r₁₂ and r₁₁=r₂₂, (iii) computing a first measured slope factor K_{a,D1} representing the slope factor of FIG. 2B for the underlying tissue as “seen” by detector D1, (iv) computing a second measured slope factor K_{a,D2} representing the slope factor of FIG. 2B for that same underlying tissue as “seen” by detector D2, and (v) computing an overall measured slope K, as the arithmetic average of K_{a,D1} and K′_{a,D2}. As illustrated in FIG. 5, the coupling efficiencies cancel out such that the measured Kₐ becomes equal to the average of the ideal slopes K′_{a,D1} and K′_{a,D2}, which is tantamount to an overall ideal slope K′ₐ. As illustrated in FIG. 6, the presence of different phase error factors is similarly obviated when r₂₁=r₁₂ and r₁₁=r₂₂, the phase error factors canceling and the overall measured phase slope Kₚ becoming equal to the average of the ideal slopes K′_{p,D1} and K′_{p,D2}, which is tantamount to an overall ideal slope K′ₚ. The resultant values of μₐ, μₛ′, and SO2 are thus independent of the coupling efficiencies and phase error factors, which is indeed a desirable result.

However, as mentioned above, in order for the system of FIG. 1 to achieve this desirable result (i.e., the obviation of source intensity differences, detector efficiency differences, and skin coupling efficiency differences) it is required that each of the left and right NIR probe patches contain a dual arrangement (see FIG. 4) of source-detector pairs for each source-detector separation distance of interest. For a single source-detector separation distance, a 2×2 arrangement (two sources, two detectors, see FIG. 4) is required for each NIR probe patch, thereby requiring a total of eight elements (four sources and four detectors) for the bilateral system. For two source-detector separation distances (for example, a “near” separation distance and a “far” separation distance), a 2×4 arrangement (two sources and four detectors, or four sources and two detectors) is required for each NIR probe patch, thereby requiring a total of twelve elements (four sources and eight detectors, or eight sources and four detectors) for the bilateral system. For three source-detector separation distances (for example, a “near” separation distance, a “mid-range” separation distance, and a “far” separation distance), a 2×6 arrangement (two sources and six detectors, or six sources and two detectors) is required for each NIR probe patch, thereby requiring a total of sixteen elements (four sources and twelve detectors, or twelve sources and four detectors) for the bilateral system. In general, for “N” distinct source-detector separation distances, a (2N+2) arrangement is required for each NIR probe patch, thereby requiring a total of 2(2N+2)=4(N+1) elements for the bilateral system.

Provided according to one preferred embodiment is an NIR cerebral oximeter comprising a unitary across-the-forehead (ATF) patch configured and dimensioned to cover both the left and right sides of the forehead simultaneously, the ATF patch comprising a lateral distribution of NIR sources and detectors including either (i) a plurality of centrally located sources and at least one detector near each of the left and right ends, or (ii) a plurality of centrally located detectors and at least one source near each of the left and right ends, wherein each of the centrally located sources or detectors is used in determining each of (i) an overall chromophore level applicable for the combined left and right sides of the brain, (ii) (ii) a left-side chromophore level separately applicable for the left side of the brain, and (iii) a right-side chromophore level separately applicable for the right side of the brain. While one or more preferred embodiments is described in terms of an across-the-forehead patch for monitoring the left and right brain hemispheres simultaneously, it is to be appreciated that the present teachings further encompass a wide variety of different probe patches capable of simultaneous monitoring of two subregions of tissue that are at least partially non-overlapping, and that the ATF forehead represents but one particularly useful example. Thus, for example, there could be provided in accordance with another preferred embodiment a user-wearable probe patch for monitoring a single kidney, where the first subregion corresponds primarily to an upper part of the kidney and the second subregion corresponds primarily to a lower part of the kidney. As another example, there could be provided in accordance with another preferred embodiment a user-wearable probe patch for monitoring both kidneys, where the first subregion corresponds primarily to a left kidney and the second subregion corresponds primarily to a right kidney.

Also provided according to a preferred embodiment is an algorithm for bilateral chromophore level monitoring based on measurements acquired using the ATF patch sources and detectors, wherein the bilateral chromophore levels are computed in a manner that obviates any coupling efficiency differences or phase error differences among the different sources and detectors, subject only to certain relaxed time-invariance assumptions for the centrally located sources or detectors (specifically, that they exhibit a constant coupling efficiency ratio and a constant phase error difference between them during the monitoring session). Advantageously, because each of the centrally located sources or detectors is involved in the individual monitoring of each of the left and right sides, bilateral monitoring is provided using a reduced number of elements as compared to the use of two separate forehead patches. Advantageously, the spatial geometry of the source/detector elements on the ATF patch provides for increased source-detector separation so that deeper penetration depths into the brain can be achieved in comparison to the use of two separate forehead patches.

As used herein, the term or subscript “whole” is used to refer to a measurement or output reading that is applicable for the combined left and right side tissue of the brain. As will be understood by a person skilled in the art, the terms “whole brain,” “left side of the brain,” and “right side of the brain” as used herein, and unless otherwise stated, refer to those portions that are forward in the skull cavity toward the forehead and reachable by a relevant portion of the NIR radiation that has been introduced into the forehead. The unitary across-the-forehead (ATF) patch can alternatively be termed a whole-forehead patch, cross-forehead patch, or total-forehead patch. Preferably, PMS (phase modulated spectrophotometry) methods are used in conjunction with the ATF sources and detectors such that the absorption coefficient and effective scattering coefficient are each computed for each of a plurality of NIR wavelengths, and absolute SO2 values are provided. However, the preferred embodiments described herein can readily be applied in CW (continuous wave) systems. For simplicity and clarity of explanation, the more general case of PMS modulation is detailed further herein.

It has been found that accurate, clinically useful, absolute, reduced source/detector bilateral SO2 monitoring based on an ATF patch according to one or more of the preferred embodiments can be achieved based on certain clinically reasonable usage and parameter assumptions. A first assumption is that there is a generally quiescent time period at the beginning of a monitoring session in which the whole brain, including both the left and right sides together, can be considered to have a generally uniform SO2 value. This assumption is particularly realistic and useful for exemplary scenarios such as surgery, in which it can be assumed that no blood clots have broken free and traveled to the brain prior to the surgery (for example), and it which case it will be particularly useful to localize which side of the brain a clot is affecting if such an event occurs during the surgery.

A second assumption is that the coupling efficiencies and phase errors of the centrally located sources (or centrally located detectors) exhibit certain time-invariance requirements that are “relaxed” in the sense that it is not strictly required that each of them remains absolutely fixed during the monitoring session. More particularly, it only needs to be assumed that the ratio of the coupling efficiencies of the centrally located sources (or centrally located detectors) remains constant during the monitoring session, and that the difference between phase errors for the centrally located sources (or centrally located detectors) remains constant during the monitoring session. These time-invariance criteria are more relaxed than a “strict” time-invariance criteria in which all coupling efficiencies and phase errors of all sources and detectors must remain fixed during the monitoring session. Notably, because the centrally located sources (or centrally located detectors) are physically nearby to each other and nestled well within the interior confines of the ATF patch, it is believed particularly realistic that the ratio of their coupling efficiencies, if not the actual values of their coupling efficiencies, will tend to remain constant throughout the monitoring session. More generally stated, one or more of the preferred embodiments described further herein is advantageously applied when it can be assumed that the particular biological volume under study has a characteristic at the beginning of the monitoring period (which can be termed a calibration period) in which both of the localized subregions (or “N” subregions if there are more than two subregions being monitored) can be considered to have a generally uniform value for the optical property to be monitored.

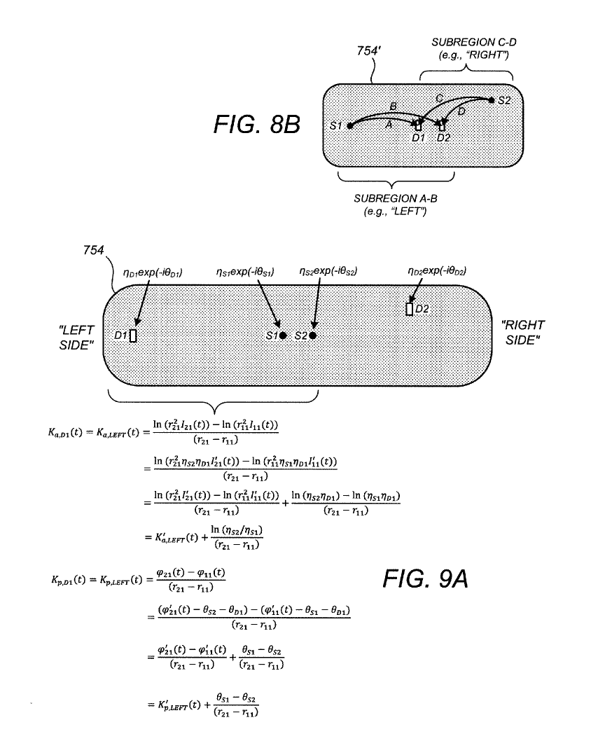

FIG. 7A illustrates an NIR cerebral oximeter 702 according to a preferred embodiment, comprising an across-the-forehead (ATF) probe patch 704 coupled via optical, electro-optical, or electrical cables 706 to a console unit 708. Console unit 708 comprises one or more optical sources 710 and optical detectors 712, each of which may be fully optical, electro-optical, or fully electrical in nature depending on the nature of the sources and detectors on the probe patch 704. For one preferred embodiment, the optical sources 710 comprise one or more laser sources, the optical detectors 712 comprise one or more photomultiplier tubes (PMTs), and the probe patch 704 consists of passive optical sources and detectors and has a general overall construction similar to one or more of the NIR probe patches disclosed in the commonly assigned and U.S. Ser. No. 12/483,610 filed Jun. 12, 2009 with the dimensions, source locations, and detector locations being as set forth herein. Console unit 708 further comprises a processor 714 coupled to control and receive information from the optical sources 710 and optical detectors 712, the processor 714 being configured, dimensioned, and programmed to achieve the functionalities described herein. Console unit 708 further comprises an output display 716 coupled to the processor 714 that simultaneously displays left, right, and whole-brain SO2 readings (and, optionally, intermediate values such as slopes, absorption coefficients, and scattering coefficients) in any of a variety of numerical and/or graphical formats. Among a variety of other control inputs, the console unit 708 further comprises a “start” button 718 that allows for user initiation of the SO2 monitoring session. The “start” button 718 can alternatively be termed a calibration button, as it instantiates a calibration process in which particular algorithm compensations (and/or other parameters) are determined based on a presumption that the optical property to be monitored is spatially homogenous throughout the different subregions of monitored tissue at that “start” time or calibration time.

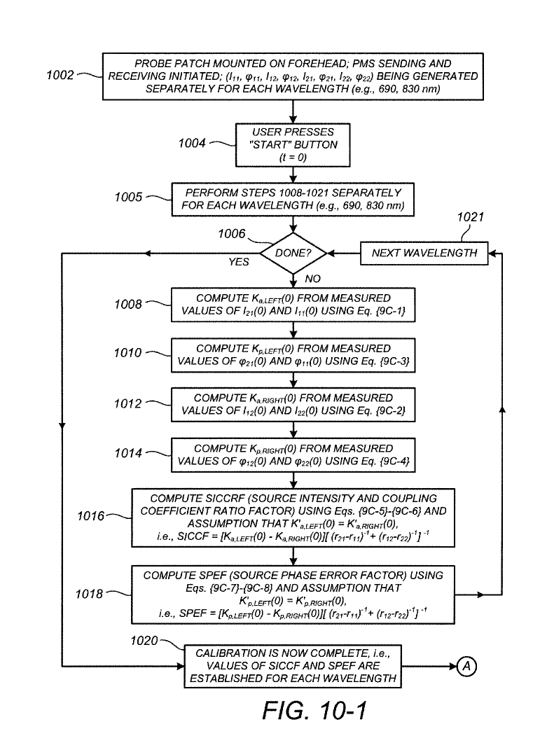

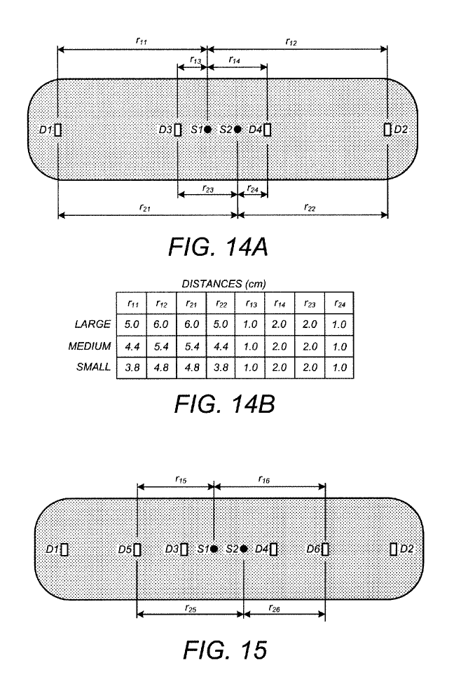

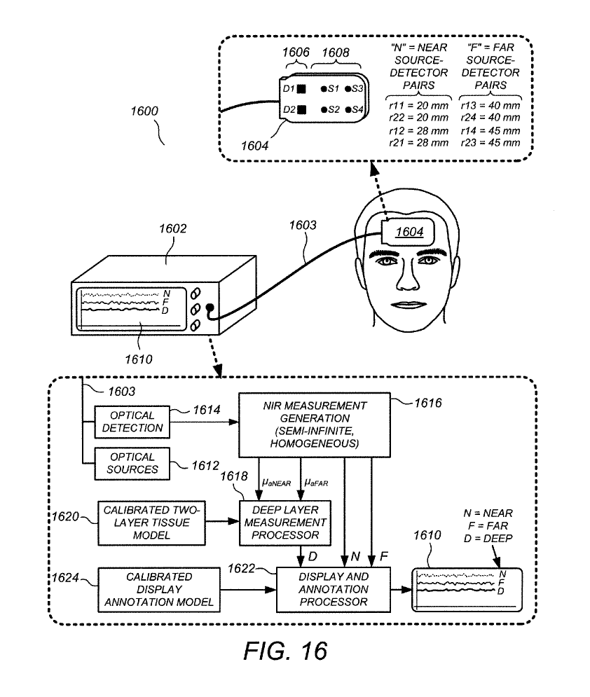

FIGS. 7B-1 and 7B-2 illustrate a simplified version of the probe patch 704 and dimensions relevant thereto according to one preferred embodiment, the probe patch 704 having only two sources S1-S2 and two detectors D1-D2 positioned as shown. Different ATF probe patches having different source-detector separation distances can be provided for differently size foreheads (e.g., large, medium, small) as illustrated in FIGS. 11A-11B, infra. In other preferred embodiments such as those illustrated in FIGS. 14A-14B and FIG. 15 infra, there are additional sets of detectors D3-D4 (FIGS. 14A-14B) and D5-D6 (FIG. 15), for providing readings that are applicable for additional source-detector separation distances. In still other preferred embodiments (not shown) there can be still more source-detector pairs provided.

FIG. 7C illustrates a simplified version of an alternative probe patch 754 that can be used in conjunction with the NIR cerebral oximeter 702 according to a preferred embodiment. Advantageously, as will be illustrated further infra, it is not required that the prior art symmetries of FIG. 4 be present in order to achieve the desired monitoring functionalities according to the preferred embodiments, and thus the probe patch 754 is shown without those symmetries present.

FIGS. 7D-1 and 7D-2 illustrate a simplified version of an alternative probe patch 755 that can be used in conjunction with the NIR cerebral oximeter 702 according to a preferred embodiment. Whereas the non-symmetric probe patch 754 still maintains a somewhat linear configuration that defines left and right subregions (albeit non-symmetrically), analogous to that of the probe patch 704, the non-symmetric probe patch 755 represents a more quadrilateral-shaped configuration that is applicable to a more compact region of tissue. For the probe patch 755, it is required only that the sources and detectors be laid out so as to define plural subregions that are at least partially non-overlapping with each other. As illustrated in FIG. 7D-2, each partially non-overlapping subregion is defined by either a single detector with two sources of differing distances therefrom (to allow the above-described slope method to be applicable) or, alternatively, a single source with two detectors of differing distances therefrom.

FIG. 7E illustrates the probe patch 755 of FIGS. 7D-1 and 7D-2 as mounted on a surface 791 of a biological volume 790, for monitoring an optical property of the subsurface tissue 792. The biological volume 790 can generally be any part of the body, and is not limited to the head of the patient.

FIG. 7F illustrates NIR cerebral oximetry based on the probe patch 755 of FIGS. 7D-1 and 7D-2, wherein there are two probe patches 755 coupled to respective sides of the forehead of the patient. For the scenario of FIG. 7F, each probe patch 755 can provide optical property readings for two subregions (e.g., an “upper” subregion and “lower” subregion, see FIG. 7D-2) for its respective hemisphere, and/or each probe patch 755 can provide a single reading for its respective hemisphere based on an averaging or other combination of the two subregions.

In keeping with the bidirectional nature of light, for each of the preferred embodiments herein there exists a converse configuration in the form of swapped source-detector positions that is also a preferred embodiment within the scope of the present teachings and that operates in essentially the same way. For example, with reference to FIG. 7B-1, an alternative converse configuration exists in which the detectors D1 and D2 are in the center of the probe patch, and the sources S1 and S2 are at the lateral peripheries of the probe patch. By way of further example, with reference to FIG. 15, infra, an alternative converse configuration exists in which each source S1, S2 is replaced by a respective detector D1, D2, and in which each detector (D1, D2, D3, D4, D5, D6) is replaced by a respective source (S1, S2, S3, S4, S5, S6). The relevant mathematical formulae and functional operation of these conversely configured preferred embodiments would be readily apparent to a person skilled in the art in view of present disclosure, and need not be discussed further herein.

For any particular ATF patch, the operational methods and computations for the different source-detector quadruplets thereon are generally independent of each other. For example, referring briefly to FIG. 14A, measurements corresponding to the S1-S2/D1-D2 quadruplet shown in FIG. 14A can be processed to compute a first absolute SO2 value, and a separate set of measurements corresponding to the S1-S2/D3-D4 quadruplet can be processed to compute a second absolute SO2 value, with there being no dependencies between the two sets of computations. Likewise, a third absolute SO2 value could be acquired for the S1-S2/D5-D6 quadruplet of FIG. 15, with no dependencies on the other two quadruplets. The multiple SO2 readings (and/or the multiple underlying values of the slopes, absorption coefficients, effective scattering coefficients, etc., at each wavelength) for the multiple source-detector quadruplets can be processed in any of a variety of different advantageous ways without departing from the scope of the present teachings. For example, in a two-quadruplet scenario (see FIG. 14A) in which there is a “near” quadruplet (S1-S2/D3-D4) and a “far” quadruplet (S1-S2/D1-D2), the “near” readings associated with lesser penetration depths can be processed in conjunction with the “far” readings associated with deeper penetration depths to extract outputs more specific to the deep brain tissue. In one preferred embodiment, the different “near” and “far” readings are processed as described in the commonly assigned Ser. No. 12/815,696, supra. Because the computations for different source-detector quadruplets are substantially the same generally independent of each other, the preferred methods for bilateral and whole-head SO2 monitoring will be detailed further herein for the simplified, single quadruplet system (S1-S2/D1-D2) of FIG. 7C.

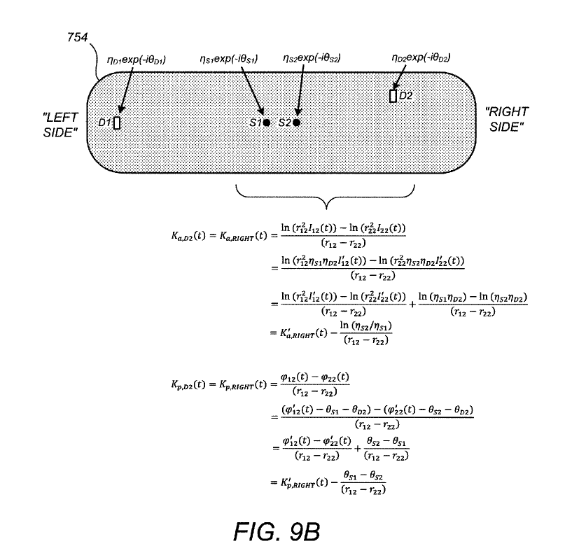

FIG. 8A illustrates near-infrared spectrophotometric (NIR) monitoring of a biological volume of a patient according to a preferred embodiment. At step 802, the NIR sources and detectors, as contained for example on the probe patch 754, are secured to a surface of the biological volume. Referring ahead briefly to FIG. 8B, in keeping with the bidirectional nature of light, there exists a converse probe patch 754′ for which the present description is equivalently applicable, in the form of swapped source-detector positions relative to the probe patch 754. Upon mounting and securing of the probe patch, a calibration interval can begin, such as by the user pressing the “start” button 718, which is followed by a monitoring interval. The calibration interval should usually last a few seconds, but can be substantially lesser or greater without departing from the scope of the present teachings. During each of a calibration interval and the subsequent monitoring interval (step 804), a first portion of light (denoted “A” in FIG. 8A) is propagated from a first optical source S1 through the medium to the first optical detector D1, a second portion of light ('B″) is propagated from the second optical source S2 through the medium to the first optical detector D1, a third portion of light (“C”) is propagated from the first optical source S1 through the medium to the second optical detector D2, and a fourth portion of light (“D”) is propagated from the second optical through the medium to the second optical detector.

At step 806, detections of the first light portion “A”, second light portion “B”, third light portion “C”, and fourth light portion “D” that were acquired during the calibration time interval are processed to compute at least one algorithm compensation that causes (i) a first result related to the optical property based on the first and second light portions “A” and “B”, which correspond to the subregion A-B (i.e., the “left” side), to be substantially equal to (ii) a second result related to the optical property based on the third and fourth light portions “C” and “D”, which correspond to the subregion C-D (i.e., the “right” side). The first and second results to which algorithm compensation is applied can be, for example, a left-side SO2 reading and a right-side SO2 reading, respectively, computed according to the “slope” method. Alternatively, the first and second results to which algorithm compensation is applied can be intermediate values, such as the intensity-based slope factor Kₐ, for the left and right sides as would be computed on the way to computing an eventual SO2 end result. Shown by way of example in FIG. 8A is a plot 850 of the SO2 results for the left side (SO2_{A-B}) and the right side (SO2_{C-D}) as would be computed by the slope method in a direct or uncompensated form based on readings taken during the calibration interval. Then, shown in FIG. 8A in the plot 851 are the results SO2_{A-B} and SO2_{C-D} as they appear in compensated form, wherein the algorithms for computing these results have been compensated in a way that forces these values to be equal.

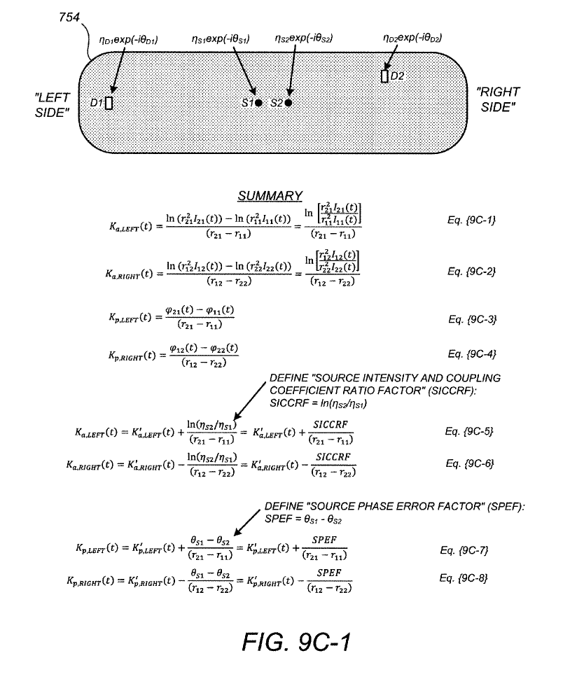

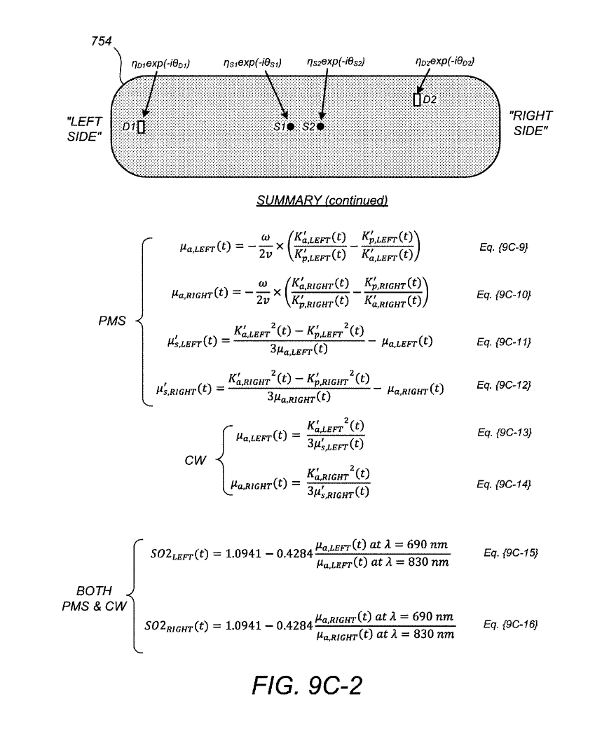

Examples of algorithm compensations applied to cause the identical results for the two respective subregions are disclosed further infra with respect to FIGS. 9A-9C and FIG. 10. For the example of FIG. 8A, the algorithm compensations are simply represented by the use of primed (′) versions of the result computation algorithms. According to one preferred embodiment, the applied algorithm compensation(s) are selected to relate to at least one non-ideality associated with one or more of the intensity of the optical sources, the sensitivity of the optical detectors, the coupling efficiency of light from the optical sources into the medium, and the coupling efficiency of light from the medium to the optical detectors. For example, one or more correction factors can be applied to change the values of the source intensity/coupling coefficients, detector efficiency/coupling coefficients, and/or phase error coefficients (for PMS implementations) used on the slope method equations such that the results of the slope method equations yield the same result for the two different subregions. Stated differently, the calibration process for a multi-subregion monitoring system according to a preferred embodiment harnesses a presumption that the optical property itself is spatially homogenous throughout the multiple subregions during the calibration interval, and that any differences between readings taken during that calibration interval are attributable to determinable non-idealities in the measurement system. The readings taken during the calibration interval are then used to determine the extent of those non-idealities and to compensate for them during the remainder of the monitoring session. Subsequently, if the multiple localized readings begin to depart from each other during the monitoring interval, those differences are indeed attributed to actual biological fluctuations in the patient (e.g., an ischemic condition in the left or right side of the brain), under a presumption that the non-idealities (or at least particular ratios related to those non-idealities, as described further infra) have remained constant during the post-calibration monitoring interval.

At step 808, subsequent to the calibration process of step 806, detections of the light portions “A” through “D” proceed throughout the monitoring interval, and the optical property is computed using the detected light in conjunction with the one or more compensation factors computed at step 806. At step 810, the resultant optical property is displayed on an output display, as illustrated by the plots 852 showing the SO2 level for the left (A-B) and right (C-D) sides of the brain, respectively. Notably, as described above in relation to step 806, it is not required that the ultimate result (in this case, SO2) be computed for each of the different subregions in determining the algorithm compensations during the calibration phase. Rather, it can be an intermediate result that is computed for each subregion (such as a slope factor), or some other property for each subregion for which homogeneity among subregions would be implicated under an assumption that the ultimate property to be measured is known to be homogeneous throughout the subregions.

FIGS. 9A-9C and FIG. 10 illustrate a particular application of the general method of FIG. 8A, in the context of a PMS-based spectrophotometry system using the probe patch 754 based on two representative wavelengths of 690 nm and 830 nm. FIGS. 9A-9C illustrate equations for adapting the slope method of absorption coefficient and effective scattering coefficient computation to a bilateral NIR cerebral oxygenation monitor using a reduced-element across-the-forehead (ATF) patch according to a preferred embodiment. FIG. 9A illustrates equations that represent the measured slopes Kₐ and Kₚ as “seen” by the left side detector D1 for the distance interval r₁₁ to r₂₁, which are denoted K_{a,LEFT}(t) and K_{p,LEFT}(t), respectively. The left-side measured slope K_{a,LEFT}(t) is computed from the measured light intensity values I₂₁(t) and I₁₁(t) as shown, while the measured left-side phase slope K_{p,LEFT}(t) is computed from the measured phase values φ₂₁(t) and φ₁₁(t) as shown. FIG. 9B illustrates equivalent equations applicable for the right side detector D2.

As illustrated in FIG. 9C, which collects and compares the slope equations from FIGS. 9A-9B, the measured left-side slope K_{a,LEFT}(t) differs from the ideal left-side slope K′_{a,LEFT}(t) only by the log of the ratio of the coupling efficiencies of the centrally located sources S1 and S2, termed herein a source intensity and coupling coefficient ratio factor (SICCRF), divided by the known quantity r₂₁−r₁₁ {Eq. 9C-5}. The measured right-side slope K_{a,RIGHT}(t) differs from the ideal right-side slope K′_{a,RIGHT}(t) only by the SICCRF (oppositely signed), divided by the known quantity r₁₂−r₂₂ {Eq. 9C-6}. Moreover, the measured left-side phase slope K_{a,LEFT}(t) differs from the ideal left-side phase slope K′_{p,LEFT}(t) only by the difference of the phase errors of the centrally located sources S1 and S2, termed herein a source phase error factor (SPEF), divided by the known quantity r₂₁−r₁₁ {Eq. 9C-7}. The measured right-side phase slope K_{p,RIGHT}(t) differs from the ideal right-side phase slope K′_{p,RIGHT}(t) only by the SPEF (oppositely signed), divided by the known quantity r₁₂−r₂₂ {Eq. 9C-8}. According to a preferred embodiment, these relationships are uniquely combined with the bilaterality assumptions set forth above (including homogeneity at time 0) to permit the separate computation of K′_{a,LEFT}(t), K′_{a,RIGHT}(t), K′_{p,LEFT}(t), and K_{p,RIGHT}(t) throughout the monitoring session, which are then used to compute separate, absolute left-side (SO2_{LEFT}(t)) and right-ride (SO2_{RIGHT}(t)) oxygen saturation values throughout the monitoring session. Briefly stated, when the user presses the “Start” button at the beginning (t=0) of the monitoring session, the algorithm compensation referenced at step 806 of FIG. 8A proceeds by a determination of the values for SICCRF and SPEF (calibrated) for each NIR radiation wavelength based on (i) measured intensity and phase values at t=0, and (ii) the assumption that K′_{a,LEFT}(0)=K′_{a,RIGHT}(0) and K′_{p,LEFT}(0)=K′_{p,RIGHT}(0). Then, for all times t>0 after the calibration is complete, the values of K′_{a,LEFT}(t), K′_{a,RIGHT}(t), K′_{p,LEFT}(t), and K′_{p,RIGHT}(t) are computed based on (i) the measured intensity and phase values at time “t”, and (ii) the determined (calibrated) values of SICCRF and SPEF.

Stated somewhat more broadly, operation of a bilateral NIR cerebral oximeter using a reduced-element ATF patch according to one preferred embodiment is based on a modified version of the slope method in which left-side slopes and right-side slopes are individually computed, wherein (i) at the quiescent beginning of the monitoring session, it is presumed that any differences in the left-side slopes versus the right-side slopes are attributable to coupling efficiency and/or phase error differences among the sources and detectors because the SO2 distribution is assumed uniform across both left and right hemispheres, and (ii) during the subsequent course of the monitoring session, it is presumed that any change in the left-side slopes or right-side slopes is attributable to timewise physical changes in the SO2 values in that hemispheres because the coupling efficiency and/or phase error differences are presumed to be fixed in time.

Notably, for the converse preferred embodiment in which the detectors D1-D2 are centrally located and the sources S1-S2 are at the left and right ends, it can be shown that the equations turn out similarly to FIG. 9C except that the source intensity and coupling coefficient ratio factor (SICCRF) becomes a detector sensitivity and coupling coefficient ratio factor (DSCCRF) equal to the log of the ratio of the coupling efficiencies of the centrally located detectors D1 and D2, and the source phase error factor (SPEF) becomes a detector phase error factor (DPEF) equal to the difference of the phase errors of the centrally located detectors D1 and D2. Thus, in the a more general expression of the preferred embodiments, the SICCRF could be replaced in the present description by a factor termed the centrally located element coupling coefficient ratio factor (CLECCRF) and the SPEF could be replaced in the present description by a factor termed the centrally located element phase error factor (CLEPEF).

FIG. 10 illustrates bilateral NIR cerebral oxygenation level monitoring according to a preferred embodiment. As the process begins at step 1002, the ATF patch has been mounted and the system has begun to acquire intensity and phase measurements during a calibration interval (the time is arbitrarily set to “0” for the time at which calibration, i.e., algorithm compensation, takes place). A set of quiescent readings for the measured intensities and measured phases is established and maintained at this time, based for example on a running 10-second averaging interval (or other suitable averaging interval) to ensure a set of smooth and reliable intensity and phase values at t=0 when the calibration process will begin. Then, with the patient in a quiescent state such that the bilateral assumptions supra are valid (e.g. the surgery operation has not yet begun and the ATF patch is safely secured to the forehead), the user presses the start button (step 1004) at time t=0 to start the calibration process, which is carried out separately for each wavelength. At steps 1008-1014, the measured slopes K_{a,LEFT}(0), K_{a,RIGHT}(0), K_{p,LEFT}(0), and K_{p,RIGHT}(0) are computed from the quiescent measured intensities and phases I₁₁(0), φ₁₁(0), I₁₂(0), φ₁₂(0), I₂₁(0), φ₂₁(0), I₂₂(0), and φ₂₂(0). At steps 1016-1018, the SICCRF and SPEF are computed based on (i) the measured slopes K_{a,LEFT}(0), K_{a,RIGHT}(0), K_{p,LEFT}(0), and K_{p,RIGHT}(0), and (ii) the assumptions that K′_{a,LEFT}(0)=K′_{a,RIGHT}(0) and K^{p,LEFT}(0) K_{p,RIGHT}(0). The calibration process for that wavelength is then complete (step 1020), and the process is repeated for each wavelength such that separate values of SICCRF and SPEF are established for each wavelength.

Subsequent to the calibration process, for all times t>0 (it can be assumed for purposes of this description that the calibration process took a negligible amount of time immediately after t=0), the known (calibrated) values of SICCRF and SPEF are used in conjunction with the ongoing measured slope values to compute the ideal slope values for the left side, right side, and whole-brain for each wavelength, which are then used as the basis for the left side, right side, and whole-brain SO2 values. Thus, at step 1024, the measured slope values K_{a,LEFT}(t), K_{a,RIGHT}(t), K_{p,LEFT}(t), and K_{p,RIGHT}(t) are computed from the measured intensities and phases at time “t”. At step 1026, the ideal slope values K′_{a,LEFT}(t), K′_{a,RIGHT}(t), K′_{p,LEFT}(t), and K′_{p,RIGHT}(t) are computed based on K_{a,LEFT}(t), K_{a,RIGHT}(t), K_{p,LEFT}(t), and K_{p,RIGHT}(t) and the values of SICCRF and SPEF. At step 1028, the absorption coefficients and effective scattering coefficients are computed from K′_{a,LEFT}(t), K′_{a,RIGHT}(t), K_{p,LEFT}(t), and K_{p,RIGHT}(t). For whole-brain monitoring, the value of K′_{a,WHOLE}(t) is computed as the average of K′_{a,LEFT}(t) and K′_{a,RIGHT}(t), the value of K′_{p,WHOLE}(t) is computed as the average of K′_{p,LEFT}(t) and K_{p,RIGHT}(t), and the corresponding absorption coefficients and effective scattering coefficients are computed therefrom at step 1029. Finally, at steps 1030-1033 the values of SO2_{LEFT}(t), SO2_{RIGHT}(t), and SO2_{WHOLE}(t) are computed from the absorption coefficients at the multiple wavelengths, and at step 1034 they are displayed on the output display 716.

Advantageously, when the bilaterality assumptions supra are valid, the absolute values SO2_{LEFT}(t), SO2_{RIGHT}(t), and SO2_{WHOLE}(t) can be reliably provided using a variety of different source-detector configurations, including asymmetric configurations. For preferred embodiments in which r₂₁−r₁₁=r₁₂−r₂₂, which is invariably the case for all of the linear source-detector arrangements above (FIGS. 7B-1, FIG. 11A), but not necessarily the case for non-linear arrangements, it is to be appreciated that above “bilaterality assumptions” (i.e., uniform SO2 at the start of the monitoring session, constant coupling efficiency ratio for the centrally located sources or detectors, constant phase error difference for the centrally located sources or detectors) are only required in order to achieve bilaterality, i.e., the individual left-brain and right-brain output readings. Importantly, the whole-brain SO2 output reading does not require time-invariance in the coupling efficiency or phase error of any source or detector on the ATF patch, nor does it require time-invariance for any of their ratios or differences. Accordingly, even in the event that the bilaterality assumptions are not valid for certain reasons or situations, the NIR cerebral oximeter is still entirely suitable as an overall, whole-brain (whole volume) SO2 monitor that is independent of coupling efficiency and phase error variations as long as r₂₁−r₁₁=r₁₂−r₂₂.

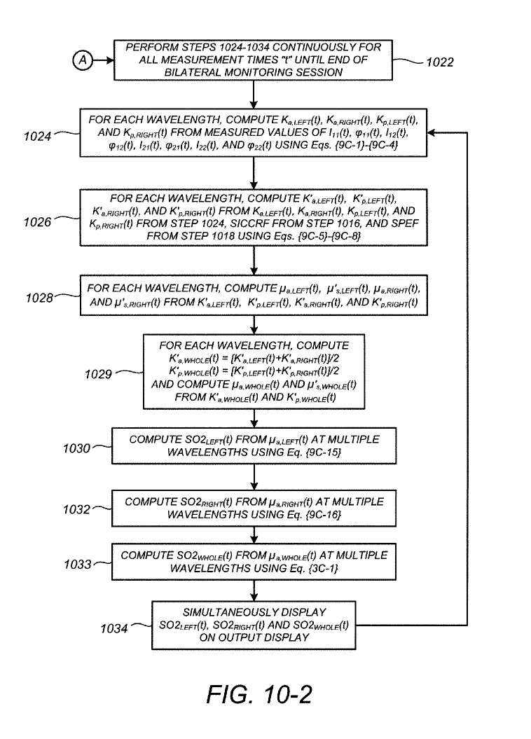

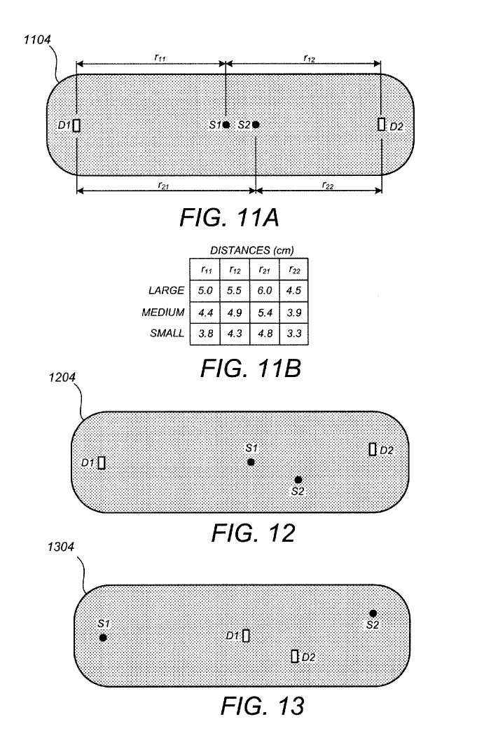

Notably, an advantageous converse functionality is provided for other preferred embodiments in which r₂₁−r₁₁≠r₁₂−r₂₂, such as for the non-linear arrangements of FIG. 7C, FIG. 7D-1, and FIGS. 12-13. In particular, for scenarios in which r₂₁−r₁₁≠r₁₂−r₂₂ but in which the above “bilaterality assumptions” are valid, the whole-head SO2 value can indeed be computed based on the relationships of FIG. 10, steps 1029 and 1033, alongside the left-side SO2 and right-side SO2 being provided. This is a useful advantage over the prior art set forth of FIGS. 4-6, which requires r₂₁−r₁₁=r₁₂−r₂₂ in order to achieve cancellation of the source-detector coupling efficiencies and phase errors.

Referring again to the preferred embodiments of FIGS. 8A, 14A, and 15, it can be seen that, for a single source-detector separation distance (FIG. 8A), only two sources and two detectors (a total of four elements) are required on the bilateral ATF patch. For two source-detector separation distances (FIG. 14A), only two sources and four detectors (a total of six elements) are required on the bilateral ATF patch. For three source-detector separation distances (FIG. 15), only two sources and six detectors (a total of eight elements) are required on the bilateral ATF patch. In general, for “N” distinct source-detector separation distances, a total of only 2(N+1) elements are required for the bilateral ATF patch, which compares favorably with the requirement of 4(N+1) elements for the dual NIR probe patch arrangement of FIG. 1, supra.

According to one preferred embodiment, each NIR cerebral oximeter described above is provided as a dual-purpose unit, having a first user-selected mode in which bilateral monitoring is provided (e.g., in controlled conditions such as surgery environments in which a clinician can reliably make the assumption of a fully-homogenous initial tissue state and time-invariant coupling efficiency/phase error differences among the sources and detectors), and having a second user-selected mode as an overall, whole-head, deep-penetration SO2 monitor. In preferred embodiments in which all of the readings (left side, right side, whole-head) are provided at all times, the clinician is able to make their own judgment about the utility of the provided readings and/or to derive insights about the patient's regional and overall brain oxygenation states based on variations of the readings and their previous clinical experiences therewith.

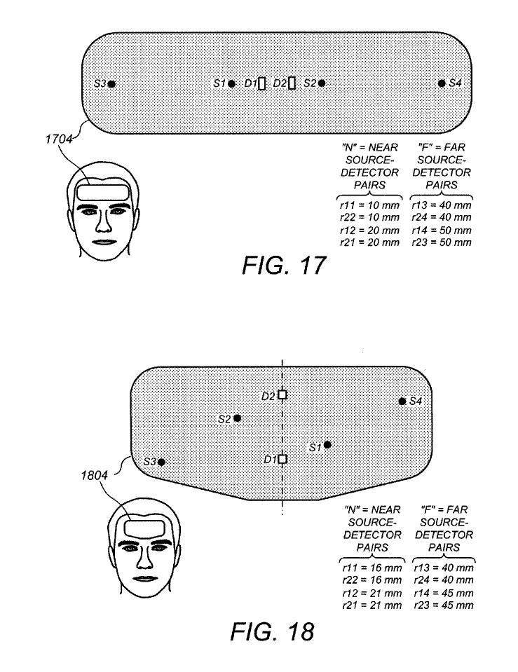

As used herein with particular relation to descriptions accompanying the preferred embodiments of FIGS. 16-29, the terms “top layer,” “outer layer”, “surface layer”, and “shallow layer” are generally used to refer to the “non-interesting” or “non-vital” layers (skin, skull, and cerebrospinal fluid layers, treating them as a single layer of material) that intervene between the NIR probe patch and the “interesting” tissue, while the term “deep layer” or “bottom layer” refers to the “interesting” tissue (the brain) that is disposed beneath the top/outer/surface/10 shallow tissue layer(s) relative to the NIR probe patch. Although the preferred embodiments are detailed herein for a two-layer tissue model of the human head, which has been found to be a particularly good level of abstraction at which to approach NIR cerebral oxygenation level monitoring from the joint perspectives of modeling, hardware implementation, and effectively displaying information to the clinician, the scope of the preferred embodiments is not so limited and would be readily extendible to tissue models of the human head having three or more layers.

FIG. 16 illustrates an NIR cerebral oximetry system 1600 according to a preferred embodiment, including a console unit 1602 coupled via an all-optical coupling cable 1603 to an NIR probe patch 1604. NIR probe patch 1604 includes sources 1608 and detectors 1606 as shown that generally establish a “far” source-detector spacing “F” and a “near” source-detector spacing “N”. The NIR cerebral oximetry system 1600 can be similar to the multi-layer NIR cerebral oximetry system of the commonly assigned Ser. No. 12/815,696, supra (hereinafter “the '696 application”) for the several preferred embodiments thereof that art directed to two-layer tissue models, the NIR cerebral oximetry system 1600 herein having further features with respect to judicious display of patient information and additional alternative methods for deep-layer SO2 computation as described further hereinbelow. In other preferred embodiments, the NIR cerebral oximetry system 1600 can be similar in many respects to the bilateral/biregional/multiregional NIR cerebral oximetry system of FIGS. 7A-15, supra, and more particularly to a multiregional system in which the different subregions monitored correspond to different penetration depths (i.e., different source-detector spacings) over the same region of tissue.

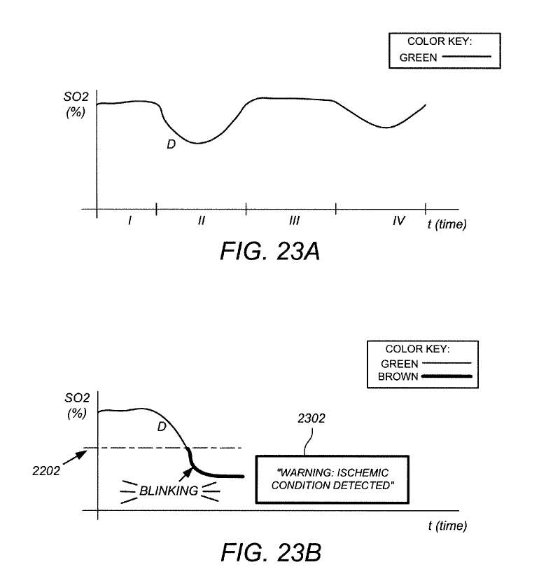

According to a preferred embodiment, coupled to the all-optical NIR probe patch 1604 is optical source hardware 1612, optical detection hardware 1614, and NIR measurement generation hardware 1616 generates PMS-based measurements and/or CWS-based measurements of the absorption coefficients μ_{aNEAR} and μ_{aFAR} (and corresponding effective scattering coefficients as needed) for the near and far source-detector spacings, computed under an assumption that the tissue is a semi-infinite, homogenous medium, similar to the manner described in the '696 application. Based on a calibrated two-layer tissue model 1620 that is detailed further in FIG. 4 of the instant specification, and that is also detailed further in the '696 application, a deep-layer measurement processor 1618 computes the absorption coefficient μ_{aNEAR} for the deep layer of tissue (and corresponding effective scattering coefficient as needed), and computes therefrom a deep layer SO2 reading “D”. The NIR measurement generation hardware 1616 also generates an SO2 reading “N” for the near source-detector spacing, and an SO2 reading “F” for the far source-detector spacing. The values of N, F, and D can be computed from the absorption coefficients (as computed for wavelengths above and below an isosbestic wavelength) according to Eqs. {19-5}-{19-7} of FIG. 19, infra.

According to one preferred embodiment, the parameters “a” and “b” at Eqs. {19-1} and {19-2} are fixed, predetermined constants that are precomputed according to a calibration process involving a large population of test phantoms or other calibration methods as described in the '696 application, supra. In another preferred embodiment, the parameters “a” and “b” can be varying with respect to particular computed parameters and/or clinical parameters, for example, they can be functions of μ_{aNEAR}, μ_{aFAR}, μ′_{sNEAR}, and/or μ′_{sFAR}, and/or they can be functions of skull thickness, according to lookup tables or other methods of determination according to a previously performed calibration process. In still another preferred embodiment, the parameters “a” and “b” can further be time-varying according to trends in the values of μ_{aNEAR}, μ_{aFAR}, μ′_{sNEAR}, and/or μ′_{sFAR} or related trends.

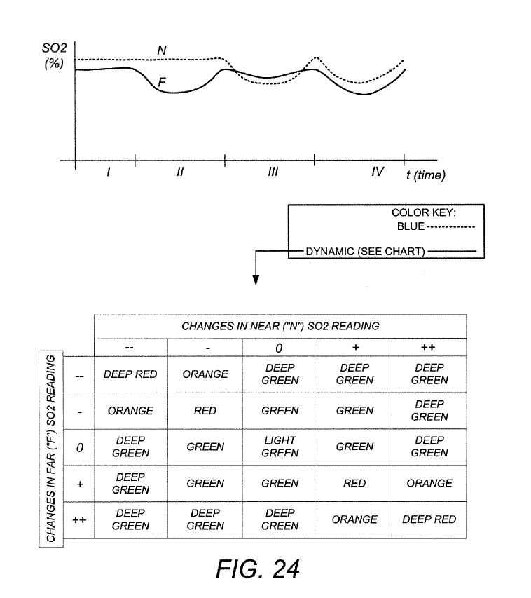

According to yet another preferred embodiment, the value of the deep-level SO2 reading “D” can be computed in a more direct process, directly from the computed SO2 values N(SO2_{NEAR}) and F (SO2_{FAR}) using the model illustrated in FIG. 20. The values of the parameters “c” and “d” in FIG. 20 can be fixed, predetermined constants, or alternatively they can they can be functions of μ_{aNEAR}, μ_{aFAR}, μ′_{sNEAR}, and/or μ′_{sFAR}, and/or functions of N and F, and/or they can be functions of skull thickness, according to lookup tables or other methods of determination according to a previously performed calibration process. In still another preferred embodiment, the parameters “c” and “d” can further be time-varying according to trends in the values of μ_{aNEAR}, μ_{aFAR}, μ′_{sNEAR}, and/or μ′_{sFAR}, and/or trends in N and F, and/or related trends.

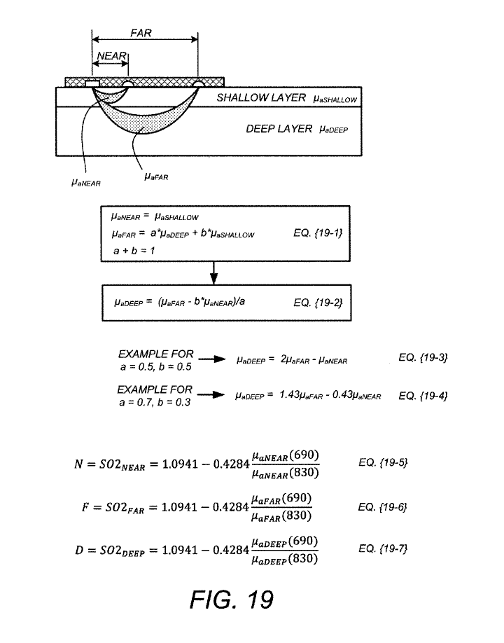

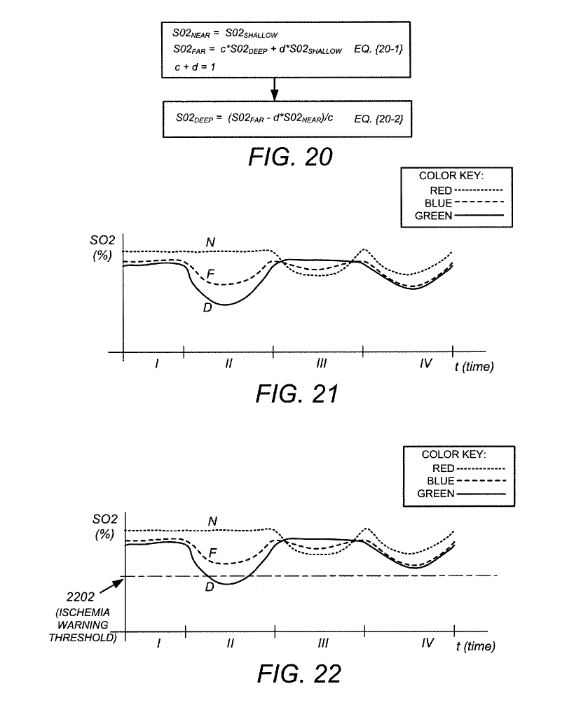

FIG. 17 illustrates an NIR probe patch 1704 that can also be used in conjunction with the NIR cerebral oximetry system 1600, which is functionally equivalent to that of FIG. 14A, supra, with sources being replaced by detectors and detectors being replaced by sources. FIG. 18 illustrates another NIR probe patch 1804 that can be used in conjunction with the NIR cerebral oximetry system 1600, which has been found to be advantageous in that the sinus cavity of the patient, and/or the separation physiology between the left and right hemispheres of the brain, has less of a differential effect on the central detectors D1 and D2. More particularly, it has been found that such differential effect, which can be more substantial in the NIR probe patch 1704 of FIG. 17, can be substantially reduced or eliminated by placing the central detectors D1 and D2 along the same vertical line in the center portion of the patch, as with the NIR probe patch 1804 of FIG. 18.

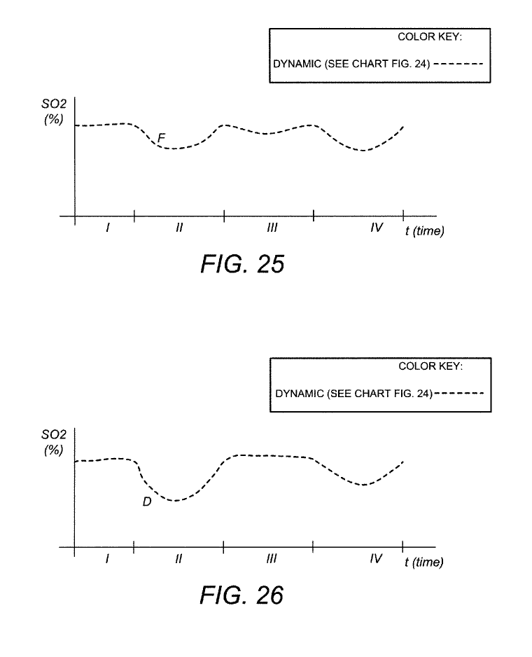

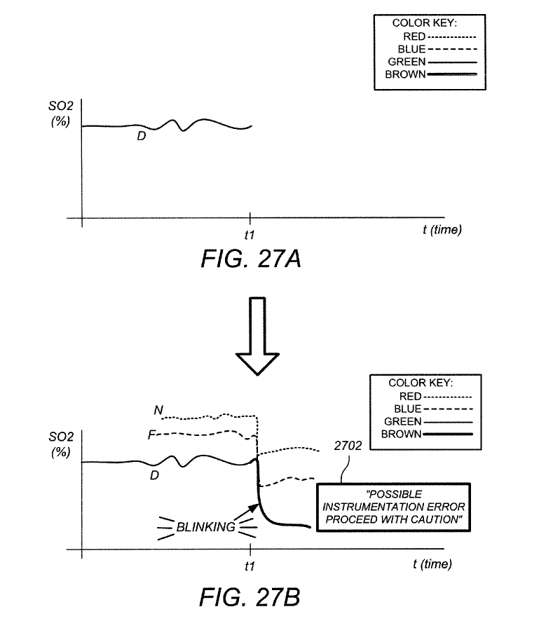

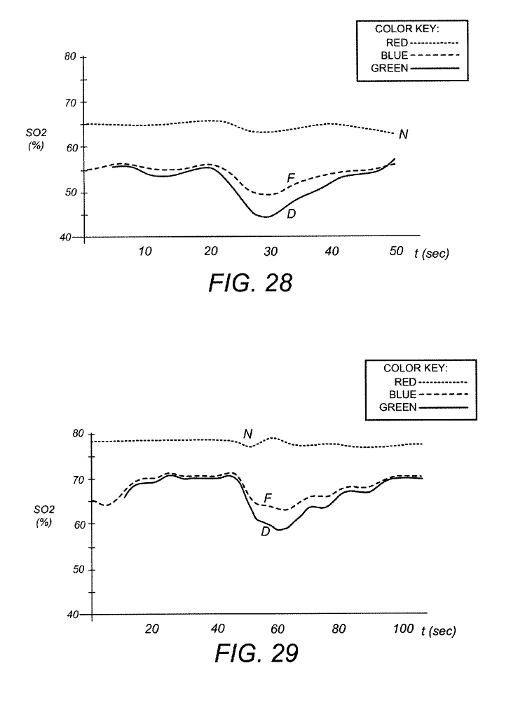

According to a preferred embodiment, the NIR cerebral oximetry system 1600 further comprises a display and annotation processor 1622 that receives the values of D, N, and F (and optionally other underlying values such as μ_{aNEAR}, μ_{aFAR}, μ′_{sNEAR}, μ′_{sFAR}, etc.) and renders onto a viewable display 1610 one or more traces and associated annotations as illustrated in one or more of FIG. 21, FIG. 22, FIG. 23A, FIG. 23B, FIG. 24, FIG. 25, FIG. 26, FIG. 27A, FIG. 27B, FIG. 28, and FIG. 29, each of which has been found to be advantageous in one or more respects. The display and annotation processor 1622 can drive the display 1610 according to a calibrated display annotation model 1624 that can be pre-calibrated based on empirical observations of the NIR cerebral oximetry system 1600 as applied to various static or dynamic phantom configurations, laboratory animals, and so forth, wherein the displays are highlighted as shown in one or more of FIG. 21, FIG. 22, FIG. 23A, FIG. 23B, FIG. 24, FIG. 25, FIG. 26, FIG. 27A, FIG. 27B, FIG. 28, and FIG. 29 under the correspondingly appropriate clinical condition.

FIG. 21 illustrates a user display which can be rendered onto the viewable display 1610 according to a preferred embodiment, comprising the three traces N, F, and D, which has been found to provide a useful level of detail without causing information overload to the clinician. Starting with a quiescent time segment I, there is an exemplary time period II in which the near reading “N” stays fixed while the far reading “F” decreases, which is more likely representative of a truly ischemic condition. In such case, when the deep SO2 level “D” is properly computed as described above using proper calibration coefficients, this condition is properly reflected (see FIG. 22) and the deep trace “D” passes below an ischemia threshold 2202. In the example of FIG. 21, there follows an exemplary time period III in which the near reading “N” decreases while the far reading “F” increases, and an exemplary time period IV in which the near reading “N” decreases and the far reading “F” both decrease. These periods III and IV are less likely to be representative of a truly ischemic condition, and when the deep SO2 level “D” is properly computed as described above using proper calibration coefficients, this condition is properly reflected (see FIG. 22) in that the deep trace “D” stays above the ischemia threshold 2202.

FIG. 23A illustrates a user display which can be rendered onto the viewable display 1610 according to another useful preferred embodiment, in which the deep trace “D” is displayed alone. Also, as shown in FIG. 23B, a visual indicator (blinking trace and warning 2302) can be displayed when there is a problematic ischemic condition.

FIG. 24 illustrates a user display which can be rendered onto the viewable display 1610 according to another preferred embodiment, in which only the near trace “N” and far trace “F” are shown, with the “F” trace color coded according to the chart shown in FIG. 24. Thus, for the example shown, the displayed color of the “F” curve turns toward red when changes in “N” and “F” tend toward levels indicative of an instrumentation error condition (one possible trigger for such behavior, for example, could be that the NIR probe patch is coming loose from the patient's forehead). FIG. 25 illustrates a user display having the far trace F alone, and FIG. 26 illustrates a user display having the deep trace D alone, each color coded according to the chart of FIG. 24, each of which can also be an effective display by itself.

FIGS. 27A-27B illustrate a time progression for a user display (i.e., an illustration of the same display at two different times) which can be rendered onto the viewable display 1610 according to another useful preferred embodiment. For the preferred embodiment of FIGS. 27A-27B, the deep trace D is displayed by itself under normal (non-error) conditions (FIG. 27A), but then the near trace N and far trace F both pop up on the display alongside the deep trace D if an error condition is detected (FIG. 27B), along with error indicators (e.g., a blinking version of the deep trace D and a warning dialog 2702). For the scenario of FIGS. 27A-27B, the clinician can quickly and intuitively see that some kind of physical disturbance (e.g., due to wires getting caught, or the patient moving, etc.) has caused a problem with probe patch coupling, for example. Finally, FIGS. 28 and 29 illustrate user displays which can be rendered onto the viewable display 1610 according to a preferred embodiment, each being similar to that of FIG. 21, supra, and corresponding to an actual laboratory experiment in which a human patient (a different patient in each of FIGS. 28 and 29) purposely hyperventilated while being monitored by an NIR cerebral oximetry system similar to the NIR cerebral oximetry system 100 of FIG. 1, supra.

Whereas many alterations and modifications of the present invention will no doubt become apparent to a person of ordinary skill in the art after having read the foregoing description, it is to be understood that the particular embodiments shown and described by way of illustration are in no way intended to be considered limiting. For example, in an alternative preferred embodiment relating to across-the-forehead (ATF) probe patches such as patches 704 and 754 of FIG. 7, supra, the above-mentioned “strict” time-invariance criteria is assumed in which all coupling efficiencies and phase errors of all sources and detectors on the ATF patch must remain fixed during the monitoring session. In another alternative preferred embodiment, the above-mentioned “strict” time-invariance criteria is assumed only for the centrally located sources or detectors on the ATF patch, and not the outlying detectors or sources. Therefore, reference to the details of the preferred embodiments are not intended to limit their scope, which is limited only by the scope of the claims set forth below.

What is claimed is:

1. A method for non-invasive spectrophotometric monitoring of an optical property of a medium, comprising:
securing each of a first optical source, a second optical source, a first optical detector, and a second optical detector to a surface of the medium;
during each of a calibration interval and a monitoring interval, said monitoring interval being subsequent to said calibration interval, propagating a first portion of light from the first optical source through the medium to the first optical detector, a second portion of light from the second optical source through the medium to the first optical detector, a third portion of light from the first optical source through the medium to the second optical detector, and a fourth portion of light from the second optical through the medium to the second optical detector;
processing detections of said first, second, third, and fourth light portions acquired during said calibration time interval to compute at least one algorithm compensation that causes a first result related to said optical property based on said first and second detected light portions to be substantially equal to a second result related to said optical property based on said third and fourth detected light portions; and
processing detections of said first, second, third, and fourth light portions acquired during said monitoring interval in conjunction with said at least one algorithm compensation to compute a monitoring result for the optical property of the medium.

2. The method of claim 1, said first and second light portions corresponding to a first subregion of the medium, said third and fourth light portions corresponding to a second subregion of the medium that is at least partially non-overlapping with said first subregion, wherein said computing said at least one algorithm compensation comprises:
(i) computing at least one error factor associated with at least one non-ideality of said optical sources and/or detectors to which a difference in said first and second results would be attributable if the optical property was known to be spatially homogenous throughout said first and second subregions during said calibration interval; and
(ii) determining at least one compensation factor associated with said at least one error factor that causes said first and second results to be substantially equal for said calibration interval.

3. The method of claim 2, wherein said at least one compensation factor remains fixed throughout said monitoring interval.

4. The method of claim 2, wherein said at least one non-ideality is associated with one or more of intensity of the optical sources, sensitivity of the optical detectors, coupling efficiency of light from the optical sources into the medium, and coupling efficiency of light from the medium to said optical detectors.

5. The method of claim 4, wherein said first and second optical sources modulate said light portions according to a phase modulated spectrophotometry (PMS) modulation pattern, and wherein said at least one non-ideality is further associated with an error in detected phase differences between corresponding light portions.

6. The method of claim 4, wherein said monitoring result for the optical property of the medium comprises (i) a first monitoring result applicable to said first subregion and based on said detected first and second light portions, and (b) a second monitoring result applicable to said second subregion and based on said detected third and fourth light portions, wherein said first and second monitoring results are computed in a manner that is substantially independent of said at least one non-ideality while requiring only said two optical sources and said two optical detectors.

7. The method of claim 6, wherein said at least one error factor and said at least one compensation factor are each computed in a manner that accommodates spatial asymmetries among the optical sources and optical detectors, whereby said computing said first and second monitoring results is substantially independent of said at least one non-ideality while not requiring spatial symmetry among the optical sources and optical detectors.

8. The method of claim 1, wherein said optical sources and detectors are positioned on a wearable patch secured to the surface of the medium, said wearable patch being generally elongate and having first and second ends and a center region therebetween, wherein said first and second optical detectors are positioned near said first and second ends, respectively, and wherein said first and second optical sources are positioned near said center region.

9. The method of claim 8, wherein said optical sources and detectors are asymmetrically positioned such that (a) a difference in distances to said first and second optical sources as respectively measured from said first optical detector is not equal to (b) a difference in distances to said second and first optical sources as respectively measured from said second optical detector.

10. The method of claim 8, wherein said medium comprises at least a portion of a cerebral volume of a patient, wherein said wearable patch is secured to the forehead of the patient, and wherein said first and second subregions correspond to opposing brain hemispheres of the patient.

11. A method for non-invasive spectrophotometric monitoring of an optical property of a medium, comprising:
securing each of a first optical source, a second optical source, a first optical detector, and a second optical detector to a surface of the medium, each being secured at a different one of a first location, a second location, a third location, and a fourth location on said surface, wherein said first and second optical sources are secured at either (i) said first and fourth locations, respectively, or (ii) said second and third locations, respectively;
during each of a calibration interval and a monitoring interval, said monitoring interval being subsequent to said calibration interval, propagating a first portion of light through the medium between said first and second locations, a second portion of light through the medium between said first and third locations, a third portion of light through the medium between said second and fourth locations, and a fourth portion of light through the medium between said third and fourth locations;
processing detections of said first, second, third, and fourth light portions acquired during said calibration time interval to compute at least one algorithm compensation that causes a first result related to said optical property based on said first and second detected light portions to be substantially equal to a second result related to said optical property based on said third and fourth detected light portions; and
processing detections of said first, second, third, and fourth light portions acquired during said monitoring interval in conjunction with said at least one algorithm compensation to compute a monitoring result for the optical property of the medium.

12. The method of claim 11, said first and second light portions corresponding to a first subregion of the medium, said third and fourth light portions corresponding to a second subregion of the medium that is at least partially non-overlapping with said first subregion, wherein said computing said at least one algorithm compensation comprises:
(i) computing at least one error factor associated with at least one non-ideality of said optical sources and/or detectors to which a difference in said first and second results would be attributable if the optical property was known to be spatially homogenous throughout said first and second subregions during said calibration interval; and
(ii) determining at least one compensation factor associated with said at least one error factor that causes said first and second results to be substantially equal for said calibration interval.

13. The method of claim 12, wherein said at least one compensation factor remains fixed throughout said monitoring interval.

14. The method of claim 12, wherein said at least one non-ideality is associated with one or more of intensity of the optical sources, sensitivity of the optical detectors, coupling efficiency of light from the optical sources into the medium, and coupling efficiency of light from the medium to said optical detectors.

15. The method of claim 14, wherein said first and second optical sources modulate said light portions according to a phase modulated spectrophotometry (PMS) modulation pattern, and wherein said at least one non-ideality is further associated with an error in detected phase differences between corresponding light portions.

16. The method of claim 14, wherein said monitoring result for the optical property of the medium comprises (i) a first monitoring result applicable to said first subregion and based on said detected first and second light portions, and (b) a second monitoring result applicable to said second subregion and based on said detected third and fourth light portions, wherein said first and second monitoring results are computed in a manner that is substantially independent of said at least one non-ideality while requiring only said two optical sources and said two optical detectors.

17. The method of claim 16, wherein said at least one error factor and said at least one compensation factor are each computed in a manner that accommodates spatial asymmetries among the optical sources and optical detectors, whereby said computing said first and second monitoring results is substantially independent of said at least one non-ideality while not requiring spatial symmetry among the optical sources and optical detectors.

18. The method of claim 11, wherein said optical sources and detectors are positioned on a wearable patch secured to the surface of the medium, said wearable patch being generally elongate and having first and second ends and a center region therebetween, wherein said first and fourth locations are positioned near said first and second ends, respectively, and wherein said second and third locations are positioned near said center region.

19. The method of claim 18, wherein said first, second, third, and fourth locations are asymmetrically positioned such that (a) a difference in distances to said second and third locations as respectively measured from said first location is not equal to (b) a difference in distances to said third and second locations as respectively measured from said fourth location.

20. The method of claim 18, wherein said medium comprises at least a portion of a cerebral volume of a patient, wherein said wearable patch is secured to the forehead of the patient, and wherein said first and second subregions correspond to opposing brain hemispheres of the patient.

21. An apparatus for non-invasive spectrophotometric monitoring of an optical property of a tissue volume of a patient, comprising:
a probe patch wearable on a surface of the tissue volume of the patient during each of a calibration interval and a monitoring interval, the probe patch comprising a first optical source, a second optical source, a first optical detector, and a second optical detector each positioned at a different one of a first location, a second location, a third location, and a fourth location on a surface-facing side of the probe patch, wherein said first and second optical sources are secured at either (i) said first and fourth locations, respectively, or (ii) said second and third locations, respectively, said probe patch being configured to propagate a first portion of light through the medium between said first and second locations, a second portion of light through the medium between said first and third locations, a third portion of light through the medium between said second and fourth locations, and a fourth portion of light through the medium between said third and fourth locations;
a calibration processor configured to process detections of said first, second, third, and fourth light portions acquired during said calibration time interval and to determine therefrom at least one algorithm compensation that causes a first result related to said optical property based on said first and second detected light portions to be substantially equal to a second result related to said optical property based on said third and fourth detected light portions;
a monitoring processor configured to process detections of said first, second, third, and fourth light portions acquired during said monitoring interval in conjunction with said at least one algorithm compensation to compute a monitoring result for the optical property of the medium; and
a display device coupled to said monitoring processor for displaying said monitoring result during said monitoring session.

22. The apparatus of claim 21, said first and second light portions corresponding to a first subregion of the tissue volume, said third and fourth light portions corresponding to a second subregion of the tissue volume that is at least partially non-overlapping with said first subregion, wherein said monitoring result for the optical property of the medium includes (i) a first monitoring result applicable to said first subregion and based on said detected first and second light portions, and (b) a second monitoring result applicable to said second subregion and based on said detected third and fourth light portions

23. The apparatus of claim 22, wherein said first, second, third, and fourth locations are asymmetrically positioned on said probe patch such that (a) a difference in distances to said second and third locations as respectively measured from said first location is not equal to (b) a difference in distances to said third and second locations as respectively measured from said fourth location.

24. The apparatus of claim 22, said probe patch being generally elongate and having first and second ends and a center region therebetween, wherein said first and fourth locations are positioned near said first and second ends, respectively, and wherein said second and third locations are positioned near said center region.

25. The apparatus of claim 24, wherein said probe patch is configured for securing to the forehead of a patient, and wherein said first and second subregions correspond to opposing brain hemispheres of the patient.

26. The apparatus of claim 22, wherein said first location is positioned relatively near to said second and third locations, wherein said fourth location is positioned relatively far from said second and third locations such that said first subregion has a relatively shallow penetration depth and said second subregion has a relatively deep penetration depth, wherein said monitoring processor is configured to:
compute a near SO₂ result corresponding to said first subregion based on said detected first and second light portions;
compute a far SO₂ result corresponding to said second subregion based on said detected third and fourth light portions; and
compute a deep SO₂ result based on a weighted difference between said near and far SO₂ results;
and wherein said display device is configured to selectively display color-coded time traces corresponding to said near, far, and deep SO₂ results.
